(19) United States
(12) Patent Application Publication
(10) Pub. No.: US 2013/0269694 A1
(43) Pub. Date: Oct. 17, 2013

(54) INHALER CONTROLLED BY MOBILE DEVICE

(71) Applicant: DANCE PHARMACEUTICALS, INC., San Francisco, CA (US)

(72) Inventors: Ryan S. Patton, San Francisco, CA (US); John Patton, San Francisco, CA (US)

(73) Assignee: Dance Pharmaceuticals, Inc., San Francisco, CA (US)

(21) Appl. No.: 13/840,588

(22) Filed: Mar. 15, 2013

Related U.S. Application Data
(60) Provisional application No. 61/624,729, filed on Apr. 16, 2012.
(60) Provisional application No. 61/624,531, filed on Apr. 16, 2012.

(51) Int. Cl. A61M 16/14

(52) U.S. Cl. CPC A61M 16/14
USPC 128/203.14

(57) ABSTRACT

An apparatus for providing nebulized medicant to a user is disclosed. The apparatus may include an air intake for drawing an air flow into the inhalation device. The apparatus may also include a medicant reservoir for receiving liquid medicant. The apparatus may further include a nebulizer for nebulizing the liquid medicant into the air flow. The apparatus may additionally include a mouthpiece for delivering the air flow with nebulized medicant to the user. The apparatus may furthermore include a communication interface configured to receive at least a first signal from a mobile device causing activation of the nebulizer.

### CROSS-REFERENCE TO RELATED APPLICATIONS

This application claims priority to U.S. Provisional Patent Application No. 61/624,729 filed Apr. 16, 2012, entitled “INHALER CONTROLLED BY MOBILE DEVICE,” and U.S. Provisional Patent Application No. 61/624,531 filed Apr. 16, 2012, entitled “METHODS FOR SUPPLYING AEROSOLIZATION DEVICES WITH LIQUID MEDICAMENTS,” the entire disclosures of which are hereby incorporated by reference, for all purposes, as if fully set forth herein.

This application is also related to U.S. patent application Ser. No. 13/004,662 filed Jan. 11 2011, entitled “PRESERVATIVE-FREE SINGLE DOSE INHALER SYSTEMS,” and U.S. patent application Ser. No. 13/004,645 filed Jan. 11, 2011, entitled “PRESERVATIVE FREE INSULIN FORMULATIONS AND SYSTEMS AND METHODS FOR AEROSOLIZING,” the entire disclosures of which are hereby incorporated by reference, for all purposes, as if fully set forth herein.

### BACKGROUND OF THE INVENTION

Drugs can be delivered to humans via a number of means, including peroral, topical, transmucosal, inhalation, and injection. When inhalation is the preferred method of delivery, inhalers of different types may be used to provide the desired delivery characteristics. These delivery characteristics can include, for example, a target concentration of the drug, a desired particle or droplet size of the drug, and a certain rate of dosage delivery. Certain drugs may be more effective in the treatment of certain conditions if the desired delivery characteristics can be closely produced by the inhalation drug delivery device.

Nebulizers are one such inhalation delivery device capable of producing tightly defined delivery characteristics. However, to do so, the nebulizer must be equipped to process required delivery characteristics and actuate the components of a nebulizer precisely to achieve the desired effect. As more particular demands are made on nebulizers available in the art, these devices may not be easily adapted to address such new demands. Embodiments of the present invention may provide solutions to these and other issues.

### BRIEF DESCRIPTION OF THE INVENTION

In one embodiment, an apparatus for providing nebulized medicant to a user is disclosed. The apparatus may include an air intake for drawing an air flow into the inhalation device. The apparatus may also include a medicant reservoir for receiving liquid medicant. The apparatus may further include a nebulizer for nebulizing the liquid medicant into the air flow. The apparatus may additionally include a mouthpiece for delivering the air flow with nebulized medicant to the user. The apparatus may furthermore include a communication interface configured to receive at least a first signal from a mobile device causing activation of the nebulizer.

In another embodiment, a method of providing nebulized medicant to a user is disclosed. The method may include receiving a first signal from a mobile device. The method may also include activating a nebulizer to nebulize a liquid medicant from a medicant reservoir in fluid communication with the nebulizer. The method may further include providing the nebulized medicant to an air flow in communication with a user's airway. The method may additionally include deactivating the nebulizer.

In yet another embodiment, a non-transitory computer readable medium having instructions stored thereon for causing an inhalation device to deliver nebulized medicant to a user is disclosed. The instructions may be executable by a processor of a mobile device for receiving an instruction from a user to activate a nebulization sequence. The instructions may also be executable for sending a first signal to a nebulizer of an inhalation device to activate the nebulizer. The instructions may further be executable for receiving feedback from the inhalation device. The instructions may additionally be executable for storing information associated with at least one of the instruction or the feedback.

### BRIEF DESCRIPTION OF THE DRAWINGS

The present invention is described in conjunction with the appended figures:

FIG. 1 illustrates an example inhalation device and mobile devices of possible embodiments of the invention;

FIG. 2 is a block diagram of one method embodiment of the invention; and

FIG. 3 is a block diagram of an exemplary computer system capable of being used in at least some portion of the apparatuses or systems of the present invention, or implementing at least some portion of the methods of the present invention.

In the appended figures, similar components and/or features may have the same numerical reference label. Further, various components of the same type may be distinguished by following the reference label by a letter that distinguishes among the similar components and/or features. If only the first numerical reference label is used in the specification, the description is applicable to any one of the similar components and/or features having the same first numerical reference label irrespective of the letter suffix.

### DETAILED DESCRIPTION OF THE INVENTION

The ensuing description provides exemplary embodiments only, and is not intended to limit the scope, applicability or configuration of the disclosure. Rather, the ensuing description of the exemplary embodiments will provide those skilled in the art with an enabling description for implementing one or more exemplary embodiments. It being understood that various changes may be made in the function and arrangement of elements without departing from the spirit and scope of the invention as set forth in the appended claims. Any detail discussed with respect to one embodiment may or may not be present in other versions of that embodiment, and/or any other discussed embodiments.

Specific details are given in the following description to provide a thorough understanding of the embodiments. However, it will be understood by one of ordinary skill in the art that the embodiments may be practiced without these specific details. For example, circuits, systems, networks, processes, and other elements in the invention may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process may be terminated when its operations are completed, but could have additional steps not discussed or included in a figure. Furthermore, not all operations in any particularly described process may occur in all embodiments. A process may correspond to a method, a function, a procedure, a subroutine, a subprogram, etc. When a process corresponds to a function, its termination corresponds to a return of the function to the calling function or the main function.

The term “machine-readable medium” includes, but is not limited to portable or fixed storage devices, optical storage devices, wireless channels and various other mediums capable of storing, containing or carrying instruction(s) and/or data. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

Furthermore, embodiments of the invention may be implemented, at least in part, either manually or automatically. Manual or automatic implementations may be executed, or at least assisted, through the use of machines, hardware, software, firmware, middleware, microcode, hardware description languages, or any combination thereof. When implemented in software, firmware, middleware or microcode, the program code or code segments to perform the necessary tasks may be stored in a machine readable medium. A processor(s) may perform the necessary tasks.

In embodiments disclosed herein, systems and methods for delivering nebulized medication to a person are provided. The systems of the invention may include inhalation devices having nebulizers capable of being controlled by software and/or hardware on assorted mobile devices, and the methods of the invention may employ such devices. The inhalation device may be similar to those described in the incorporated patents and/or patent applications, or those described herein or elsewhere, and may be configured to be controlled via a communications interface by a mobile device. The mobile device may be a cellular phone (also referred to herein as a mobile phone), a personal data assistant (PDA), a tablet computer, or other portable electronic device. In some embodiments, while instructions or control of the inhalation device may originate from the mobile device, power for a nebulizer of the inhalation device may be provided in the inhalation device itself In other embodiments, power may be provided from the mobile device or other source. The mobile device may be capable of logging usage of the inhalation device, including instructions sent to the inhalation device, and data received back therefrom.

In one embodiment, an apparatus (also referred to herein as an inhalation device) for providing nebulized medicant to a user is disclosed. The apparatus may include an air intake for drawing an air flow into the inhalation device. The apparatus may also include a medicant reservoir for receiving liquid medicant. Dry medicant may also be nebulized from the medicant reservoir, and whenever liquid medicant is discussed herein, the same embodiments may be applied to dry medicant usage. The apparatus may further include a nebulizer for nebulizing the liquid medicant into the air flow. The apparatus may additionally include a mouthpiece for delivering the air flow with nebulized medicant to the user. The apparatus may furthermore include a communication interface configured to receive at least a first signal from a mobile device causing activation of the nebulizer.

The nebulizer may include a vibrating mesh which vibrates when current/voltage is applied to a connected or included mechanism, for example a piezoelectric actuator. This vibration causes nebulization of a fluid in communication with the mesh. A power source, either on the inhalation device or elsewhere (for example, the mobile phone) provides the current/voltage. The amount and timing of current/voltage which is applied to the nebulizer may be controlled by the mobile device, which is in communication with the inhalation device. In this manner, control functions of the inhalation device are removed from inhalation device itself and provided by the mobile device. While in many embodiments the power source may still reside in the inhalation device, in other embodiments power from the mobile device or elsewhere may be provided.

In another embodiment, a method of providing nebulized medicant to a user is disclosed. The method may include receiving a first signal from a mobile device. The method may also include activating a nebulizer to nebulize a liquid medicant from a medicant reservoir in fluid communication with the nebulizer. The method may further include providing the nebulized medicant to an air flow in communication with a user's airway. The method may additionally include deactivating the nebulizer. This and other methods disclosed herein may be conducted with the inhalation devices and/or mobile devices discussed herein.

In yet another embodiment, a non-transitory computer readable medium having instructions stored thereon for causing an inhalation device to deliver nebulized medicant to a user is disclosed. The instructions may be executable by a processor of a mobile device for receiving an instruction from a user to activate a nebulization sequence. The instructions may also be executable for sending a first signal to a nebulizer of an inhalation device to activate the nebulizer. The instructions may further be executable for receiving feedback from the inhalation device. The instructions may additionally be executable for storing information associated with at least one of the instruction or the feedback. The above instructions, and instructions for conducted other methods discussed herein, may be stored in a storage medium of a mobile device. Certain complimentary instructions to achieve such functionality (for example, sending of data to be received by the mobile device), may be stored on a storage medium of the inhalation device.

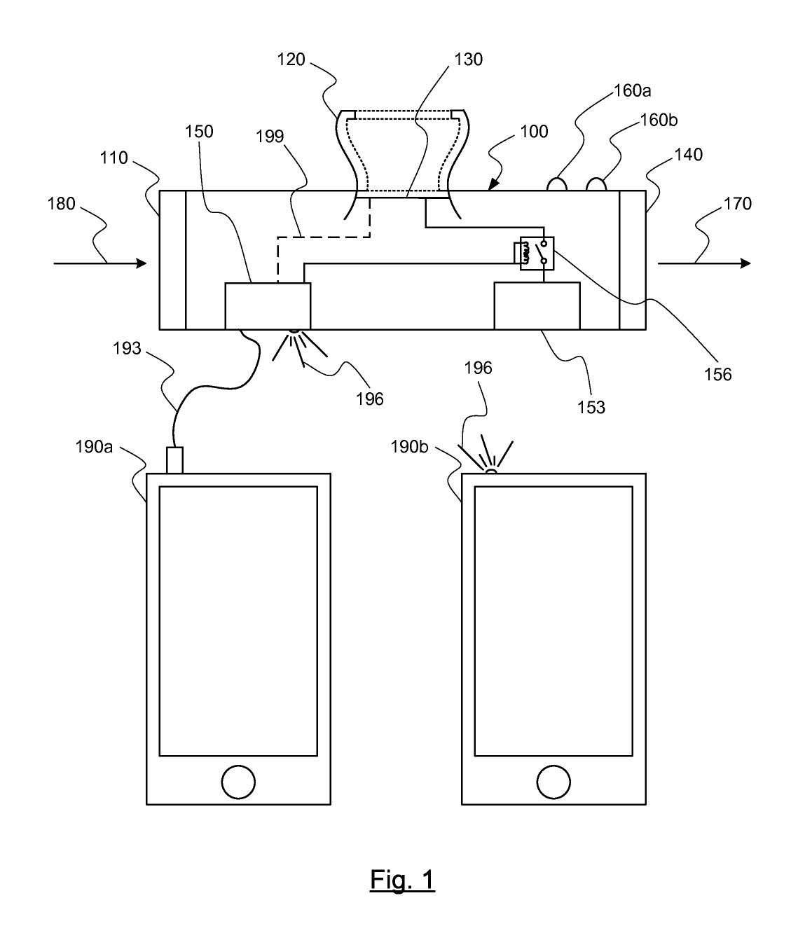

FIG. 1 shows one example inhalation device 100 disclosed in one embodiment. Inhalation device 100 includes an air intake 110, a medicant reservoir 120, a nebulizer 130, a mouthpiece 140, a communication interface 150, power supply 153, actuator 156 (shown in this example as a relay), and indicators 160. When a user draws air through inhalation device 100 at mouthpiece 140, as shown by arrow 170, air is drawn into air intake 110, as shown by arrow 180. This air passes by nebulizer 130 which, when activated via power supply 155 and actuator 156, nebulizes medicant in medicant reservoir 120 into the air stream to be received by the user. Communication interface 150 may receive instructions from a mobile device 190a (shown in this example as a cellular phone) via cable 193. Alternatively, communication interface 150 may receive instructions from a mobile device 190b (also shown in this example as a cellular phone) via wireless communications 196.

Though in this embodiment mobile device 190 is shown physically separated from inhalation device 100, in other embodiments, inhalation device 100 may be physically mounted to mobile device 190 once communication interface 150 is connected to mobile device 190. In either case, instructions from mobile device 190 would cause power to be supplied from power supply 153 to nebulizer 130 via control of actuator 156. Though actuator 156 is shown in this example as a simple relay, capable of turning on/off power to nebulizer 130, in other embodiments, actuator 156 may allow for control not only of on/off activation, but also of the level of voltage/current supplied from power supply 153 to nebulizer 130. Also, in other embodiments where power is supplied by mobile device 190, communication interface 150 may be directly coupled with nebulizer 130 (as shown by line 199 and include an actuation system therein or coupled there-between.

Depending on the embodiment, power supply 153 and/or inhalation device 100 may be disposable. In some embodiments, power supply 153 may be rechargeable, possibly from a wall-source or mobile device 190 via communication interface 150. In other embodiments, inhalation device 100 may be powered wirelessly from mobile device 190 or some other source.

Sensors, not shown, may also be present in inhalation device 100 to provide feedback to the user and/or mobile device 190. For example, a pressure transducer may be present to provide for pressure measurement of air flowing through the device, or a sensor may be placed in such components as nebulizer 130, communication interface 150, power supply 153, or actuator 156 to provide information regarding past and current operation. These sensors would be in communication with communication interface 150 so that such information could be relayed back to mobile device 150. Additionally, indicators 160, shown in this example as LED/LCD lights, could visually communicate data from sensors to the user.

In embodiments where there is a physical communication connection between communication interface 150 and mobile device 190, the connection may be a TS, TR, TRS, or TRRS connection or connector cable (also referred to as a headphone or audio cable having a conductive plug for insertion into a conductive jack). Other types of cables may also be possible, including USB (including mini-USB), FireWire (IEEE 1394), or proprietary interface cables. Cable 193 may also carry power and other actuation and/or sensor data signals to/from inhalation device 100.

In some embodiments, multiple cables may be coupled with inhalation device 100. In some embodiments, a selection of cables may be available, each having a universal end attachable to inhalation device 100, with various other ends with a particular cable plug adapted for one or more particular mobile devices 190. In some embodiments, a cable extending from inhalation 100 device may have a selection of adapters at the end available to mobile device 190.

In some embodiments, inhalation device 100 may communicate with mobile device 190 over a wireless communication. Merely by way of example, WiFi and/or Bluetooth connections may be established between inhalation device 100 and mobile device 190 to provide the same communication capabilities as the wired versions discussed above.

Software may be downloaded or loaded onto mobile devices 190 to control inhalation device 100 via communication interface 130. The downloading of software may occur in the same fashion as other application software is downloaded to the mobile device (wirelessly, or wired via PC applications). In some embodiments, the mobile device may be able to accept updates to the control software via a wired or wireless connection to another processing system and/or network. These updates may be pushed down to the mobile device and/or pulled by the mobile device, possibly at request of the user. The mobile device control software, in addition to issuing control signals to inhalation device 100, may also retrieve information from inhalation device 100, and/or track and store information related to usage of the inhalation device 100 and/or control software on mobile device 190 by a user.

The software on mobile device 190 may be configured to provide a plurality of features when controlling inhalation device 100. Merely by way of example, the mobile device allow a user to activate or deactivate the inhalation device; provide power to the inhalation device when activated; store in memory the times and duration of activation, as well as sensed pressures or other characteristics of the activation period; provide visual and/or audio guidance to the user regarding whether to breath quicker or slower through the inhalation device; inform the user, via display or audio, when medicant delivery is complete; track and store previous administrations of medicant delivery; display or play via audio present and past usage information; and transmit present and past usage information to third parties (e.g., doctors).

In some embodiments, stored information on mobile device 190 from previous use of inhalation device 100 may be used to determine characteristics of future operation of inhalation device 100. Control signals from mobile device 190 to inhalation device 100 may thereafter instruct inhalation device 100 in accordance with the determined characteristics of future operation. Merely by way of example, mobile device 100 may determine from stored data that a certain amount of medicant has been delivered to a user over a particular time period and only a certain amount is still required to be delivered per a previously set requirement (i.e., prescription). Control signals may then be issued by mobile device 190 to that effect.

In some embodiments, data related to usage of inhalation device 100 may be stored on a storage medium of mobile device 190. In there or other embodiments, a storage medium on inhalation device 100, possibly accessible by mobile device 190, may store usage data for later retrieval. In other embodiments, data may be stored in a remote location via a wired or wireless network (i.e., a remote server, a remote Internet accessible storage location, The Cloud, etc.). This data may be made available to third parties (e.g., doctors), possibly by input of electronic contact information into a software query on mobile device 100. The data being made available may mean that the data is pushed out as soon as it is available (e.g., immediately after, or even during, administration of a particular dose), or that the data is pull-able after an authorized request from a third party.

While many embodiments may use a display device and/or audio device on mobile device 190 to instruct and provide output to the user, some embodiments will incorporate indicator lights 160 on inhalation device 100 itself. This may be particularly useful in embodiments where mobile device 190 is physically coupled directly to inhalation device 100. Indicator lights 160 in these embodiments may be necessary because it will be difficult for the user to read the display screen of mobile device 100 during use. Examples of indicator lights include breathing instruction lights and/or end of dosage lights.

In these embodiments, inhalation device 100 may be shaped to firmly couple, possibly by interference fit, with mobile device 190. In other embodiments, the friction resulting from coupling of the electrical jack of mobile device 190 with the electrical plug of inhalation device 100 may be sufficient to firmly couple the two devices together.

In the manner described then, the production costs of inhalation devices 100 can be reduced by using a mobile device 190 that a user may already own to provide processing circuitry for inhalation device 100. Additionally, given the common wired and wireless communication features of most mobile devices 190, communication with a caregiver or doctor regarding compliance by the user/patient with dosage and frequency instructions may be improved. Particular ailments which could be beneficially addressed by embodiments of the instant invention include asthma, chronic obstructive pulmonary disease (COPD), lung infections, and diabetes.

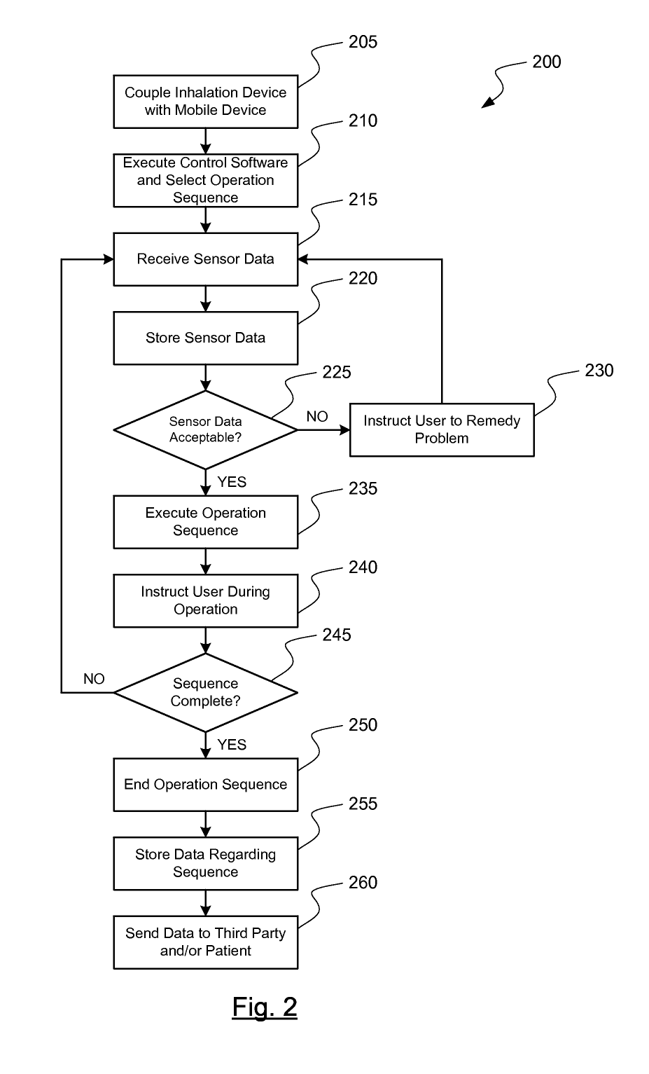

FIG. 2 is a block diagram illustrating an exemplary method 190 of the invention. At block 205, a user couples inhalation device 100 with mobile device 190, thereby establishing a communication and/or power connection between the two devices. At block 210, the user executes control software on mobile device 190 and selects an operation sequence which may specify durations, intervals, or other characteristics under which nebulizer 130 should be activated. At block 215, mobile device 190 receives sensor data from inhalation device 100. At block 220, the sensor data is stored, possibly at mobile device 190.

At block 225, mobile device 190 determines if the sensor data is within acceptable ranges as specified by the selected operating sequence and/or other requirements (potentially particular requirements depending on the model of the inhalation device 100). For example, mobile device 190 will determine if the user is pulling an adequate amount of airflow through inhalation device 100. If the sensor data is not acceptable to start operation of inhalation device 100 (e.g., low air flow), then at block 230 mobile device 190 instructs the user, visually and/or audibly, how to correct the problem (e.g., “increase airflow by breathing deeper”). The method then continues at block 215.

However, once sensor data has been determined to be in-range at block 225, at block 235, depending on the operation sequence selected by the user, at block 215, mobile device 190 sends a first signal or communication to mobile device 100 to activate nebulizer 130. At block 240, mobile device 190 continues to issue, visually and/or audibly, instructions to the user, and/or an indication that nebulization is proceeding as desired. At block 245, mobile device 190 determines if the operation sequence is complete. This may occur because of data received from sensors in inhalation device 100, or due to the parameters of the selected operating sequence. If the operation sequence is not complete, the method continues at block 215.

If the operation sequence is complete, then at block 250 mobile device 190 ends the operation sequence. This may occur either by ceasing transmission of a signal from mobile device 190 to inhalation device 100, or by transmission of a separate signal from mobile device 190 to inhalation device 100. At block 255, any end-of-sequence information or summary of information may be stored by mobile device 190. In some embodiments, at block 260, the stored data regarding the operation sequence may be sent from mobile device 190 to a third party, for example a doctor of the user, and/or the patient, possible at the request of the party. A web interface may be provided to access and/or provide this information, as well as applications for mobile devices.

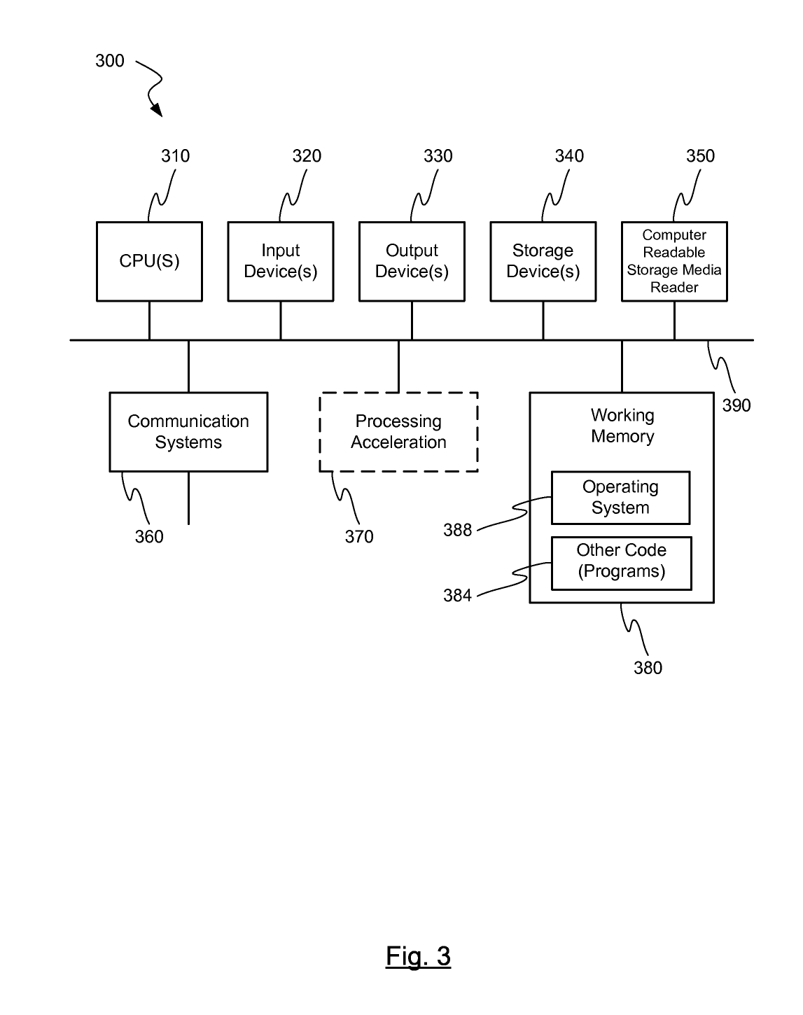

FIG. 3 is a block diagram illustrating an exemplary computer/processing system 300 in which embodiments of the present invention may be implemented. This example illustrates a computer system 300 such as may be used, in whole, in part, or with various modifications, to provide the functions of inhalation device 100, mobile device 190, and/or other components of the invention such as those discussed above. For example, various functions of mobile device 190 may be controlled by the computer system 300, including, merely by way of example, controlling nebulizer 130 of the inhalation device 100, tracking inhalation device 100 usage, transmitting usage history information to third parties, etc.

The computer system 300 is shown comprising hardware elements that may be electrically coupled via a bus 390. The hardware elements may include one or more central processing units 310, one or more input devices 320 (e.g., a mouse, a keyboard, etc.), and one or more output devices 330 (e.g., a display device, a printer, etc.). The computer system 300 may also include one or more storage device 340. By way of example, storage device(s) 340 may be disk drives, optical storage devices, solid-state storage device such as a random access memory (“RAM”) and/or a read-only memory (“ROM”), which can be programmable, flash-updateable and/or the like.

The computer system 300 may additionally include a computer-readable storage media reader 350, a communications system 360 (e.g., a modem, a network card (wireless or wired), an infra-red communication device, Bluetooth™ device, cellular communication device, etc.), and working memory 380, which may include RAM and ROM devices as described above. In some embodiments, the computer system 300 may also include a processing acceleration unit 370, which can include a digital signal processor, a special-purpose processor and/or the like.

The computer-readable storage media reader 350 can further be connected to a computer-readable storage medium, together (and, optionally, in combination with storage device(s) 340) comprehensively representing remote, local, fixed, and/or removable storage devices plus storage media for temporarily and/or more permanently containing computer-readable information. The communications system 360 may permit data to be exchanged with a network, system, computer and/or other component described above.

The computer system 300 may also comprise software elements, shown as being currently located within a working memory 380, including an operating system 384 and/or other code 388. It should be appreciated that alternate embodiments of a computer system 300 may have numerous variations from that described above. For example, customized hardware might also be used and/or particular elements might be implemented in hardware, software (including portable software, such as applets), or both. Furthermore, connection to other computing devices such as network input/output and data acquisition devices may also occur.

Software of computer system 300 may include code 388 for implementing any or all of the function of the various elements of the architecture as described herein. For example, software, stored on and/or executed by a computer system such as system 300, can provide the functions of inhalation device 100, mobile device 190, and/or other components of the invention such as those discussed above. Methods implementable by software on some of these components have been discussed above in more detail.

Embodiments of the invention have now been described in detail for the purposes of clarity and understanding. However, it will be appreciated that certain changes and modifications may be practiced within the scope of the appended claims.

What is claimed is:

1. An apparatus for providing nebulized medicant to a user, the apparatus comprising:
an air intake for drawing an air flow into the inhalation device;
a medicant reservoir for receiving liquid medicant;
a nebulizer for nebulizing the liquid medicant into the air flow;
a mouthpiece for delivering the air flow with nebulized medicant to the user; and
a communication interface configured to receive at least a first signal from a mobile device causing activation of the nebulizer.

2. The apparatus for providing nebulized medicant to a user of claim 1, wherein the mobile device comprises:
a cellular phone.

3. The apparatus for providing nebulized medicant to a user of claim 1, the apparatus further comprising:
a power supply for activating the nebulizer.

4. The apparatus for providing nebulized medicant to a user of claim 1, wherein the communication interface comprises:
a wireless communication system.

5. The apparatus for providing nebulized medicant to a user of claim 1, wherein the communication interface comprises:
a conductive plug.

6. The apparatus for providing nebulized medicant to a user of claim 5, wherein:
the apparatus is at least partially mounted to the mobile device when the conductive plug is inserted into a conductive jack of the mobile device.

7. The apparatus for providing nebulized medicant to a user of claim 1, wherein:
the first signal causes activation of the nebulizer at a certain frequency, for a certain amount of time, or at particular intervals.

8. The apparatus for providing nebulized medicant to a user of claim 1, wherein:
the communication interface is further configured to send a second signal to the mobile device.

9. The apparatus for providing nebulized medicant to a user of claim 8, wherein the second signal comprises a selection from a group consisting of:
a ready message;
an error message;
a delivery-in-process message;
a low air flow message;
an acceptable range of air flow message;
a high air flow message;
a low medicant supply message;
a historical dosage information message; and
a medicant delivery complete message.

10. The apparatus for providing nebulized medicant to a user of claim 1, wherein:
the apparatus is configured to receive power from the mobile device to activate the nebulizer.

11. The apparatus for providing nebulized medicant to a user of claim 10, wherein:
the communication interface is further configured to receive power from the mobile device.

12. The apparatus for providing nebulized medicant to a user of claim 1, wherein:
the nebulizer is configured to deactivate when no liquid medicant remains in the medicant reservoir.

13. A method of providing nebulized medicant to a user, the method comprising:
receiving a first signal from a mobile device;
activating a nebulizer to nebulize a liquid medicant from a medicant reservoir in fluid communication with the nebulizer;
providing the nebulized medicant to an air flow in communication with a user's airway; and
deactivating the nebulizer.

14. The method of providing nebulized medicant to a user of claim 13, wherein the first signal selectively couples and decouples the nebulizer to a power supply over time.

15. The method of providing nebulized medicant to a user of claim 13, wherein:
deactivating the nebulizer is caused in response to the first signal.

16. The method of providing nebulized medicant to a user of claim 13, wherein:
deactivating the nebulizer is caused in response to a second signal.

17. The method of providing nebulized medicant to a user of claim 13, wherein the method further comprises:
receiving power for the nebulizer from the mobile device.

18. A non-transitory computer readable medium having instructions stored thereon for causing an inhalation device to deliver nebulized medicant to a user, the instructions executable by a processor of a mobile device for at least:
receiving an instruction from a user to activate a nebulization sequence;
sending a first signal to a nebulizer of an inhalation device to activate the nebulizer;
receiving feedback from the inhalation device; and
storing information associated with at least one of the instruction or the feedback.

19. The non-transitory computer readable medium having instructions stored thereon for causing an inhalation device to deliver nebulized medicant to a user of claim 18, the instructions further executable by a processor of a mobile device for at least:
determining a power level of at least one of the mobile device or the inhalation device;
determining, based on determining the power level, whether to activate the nebulizer with power from the mobile device or the inhalation device.

20. The non-transitory computer readable medium having instructions stored thereon for causing an inhalation device to deliver nebulized medicant to a user of claim 18, wherein storing information associated with at least one of the instruction or the feedback comprises:
storing a log of times and amounts of medicant delivered.
